(19) United States
(12) Patent Application Publication
(10) Pub. No.: US 2005/0049631 A1
(43) Pub. Date: Mar. 3, 2005

(54) Acupressure device

(76) Inventors: Michael Carter-Smith, Surrey (GB); Roger Philip Whitaker, Surrey (GB)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/730,250

(22) Filed: Dec. 9, 2003

(30) Foreign Application Priority Data
Aug. 28, 2003 (GB) 0320159.7

(51) Int. Cl. A61B 17/00

(52) U.S. Cl. 606/204

(57) ABSTRACT

An acupressure ring for use on the outer finger, comprises a ring having a visual indicator visible when the ring is worn and a projection extending inwardly from the inner surface of the ring, the projection being approximately orthogonal to the visual indicator. Such a ring can be used to reduce or eliminate snoring.

The present invention relates to a novel acupressure device in the form of a ring designed to apply pressure at a precise location in accordance with the known techniques of acupressure.

It is well known that impairment of breathing, especially when asleep, is extremely dangerous. When asleep, impaired breathing is often indicated by snoring.

Although snoring has often been the subject of humour, it can be a sign of a more serious underlying problem, which can be dangerous or fatal to the snorer. Recent studies [e.g. Chen M L, Lin L C, Wu S C, “The effectiveness of acupressure in improving sleep” Biol. Sci. Med. Sci. (1999)] have shown that acupressure can be effective in improving sleep patterns and preventing snoring. By applying the techniques of acupressure, we have discovered a simple device which, when used correctly can reduce, or even eliminate, snoring.

Acupressure is based upon bioenergetic meridian lines that flow through the body from the fingertips and toes to the brain. There are twelve main meridians in the body connected by collaterals, these are split into six Yin and six Yang plus two, with each collateral connecting the brain to an organ associated with the specific meridian. These energy meridians can get blocked. However, the application of acupressure to the corresponding acupoint frees the obstruction by stimulating the flow of chi.

Acupressure involves applying pressure to certain meridian points on the body, including the fingers and hand, to relieve pain, stimulate bodily functions and enable muscle fibres to elongate and relax. This invention is designed to create the correct bioenergetic effect on the meridian line directly affecting the nose and throat, thereby promoting noiseless quality sleep patterns, with no or reduced snoring.

The traditional Chinese method involved the use of natural materials to create a tourniquet and dried rice pods which acted as acupressure stimulators. More recently, several acupressure rings have been developed to emulate the traditional acupressure process by blood circulation enhancement. For example, such a ring is described in US2003032978. This describes a ring with a plurality of projections extending from an inner surface thereof to apply pressure to points on an outer (“little”) finger. Other known rings are similar but have only a single, inwardly extending projection.

However, it is essential that, in order to achieve the desired result, pressure should be applied to a very precise position on the meridian line. The prior art rings do not provide any means to enable the user to ensure that they are properly positioned and so tend to be positioned haphazardly on the fingers. Random selection of pressure points will not permit the desired stimulation process on the meridian line. The key pressure point (acupoint) is quite specific to the outer finger of each hand, left (female) or right (male).

In accordance with one aspect of the present invention, there is provided an acupressure ring for use on the outer finger, and comprising a ring having a visual indicator visible when the ring is worn and a projection extending inwardly from the inner surface of the ring, the projection being approximately orthogonal to the visual indicator.

Preferably the mid-point of the projection is approximately 90° around the ring from the mid-point of the visual indicator, e.g. within ±20°, more preferably ±15° and still more preferably ±10°. The mid-points are determined along a line extending circumferentially around the ring—where appropriate, all measurements are taken in this way herein.

The projection may take a variety of shapes, for example it may be: triangular, polygonal, polyhedral, rhomboidal, wedge-shaped, quadrangular, cylindrical, square, cubic, rectangular, conical, bulb-shaped, tubular, pyramidal, spring-shaped, star-shaped, barrel-shaped, crescent-shaped, bell-shaped, egg-shaped, ellipsoidal, mushroom-shaped, balloon-shaped, bullet-shaped, capsule-shaped, apple-shaped, onion-shaped, diamond-shaped, pipe-shaped, rolling pin-shaped, horn-shaped, bottle-shaped, rose-shaped, flower head-shaped, ovoid, pear-shaped, heart-shaped, coil-shaped, hoop-shaped, turban-shaped, knot-shaped, tyre-shaped, sports bat-shape, sports racquet-shaped, prism-shaped, pie-shaped, animal-shaped, vegetable-shaped, bird-shaped, fish-shaped, insect-shaped, tool-shaped, lozenge-shaped, “z”-shaped, shaped as another raised letter of the alphabet, for example a letter from A to X, crown-shaped, bone-shaped, shaped as a transportation image (for example as a car, pedal cycle, bus, tram, train, boat, ship, aircraft, motorcycle), clover-shaped, shamrock-shaped, leek-shaped, feather-shaped, thistle-shaped, leaf-shaped, pen-shaped, coin-shaped, chain link-shaped, billiard/snooker/pool cue-shaped, brush-shaped, spoon-shaped, book-shaped, key-shaped, or tea/coffee pot-shaped. Of these, we especially prefer that they should be round, cylindrical, conical, square or rectangular. Alternatively, the projection may have an essentially conical, preferably truncated conical, shape. The shapes described refer to the shape impressed in the finger when the ring is in place on an outer, little, finger.

If desired, there may be more than one such projection. However, it is generally preferred that there should be a single projection only.

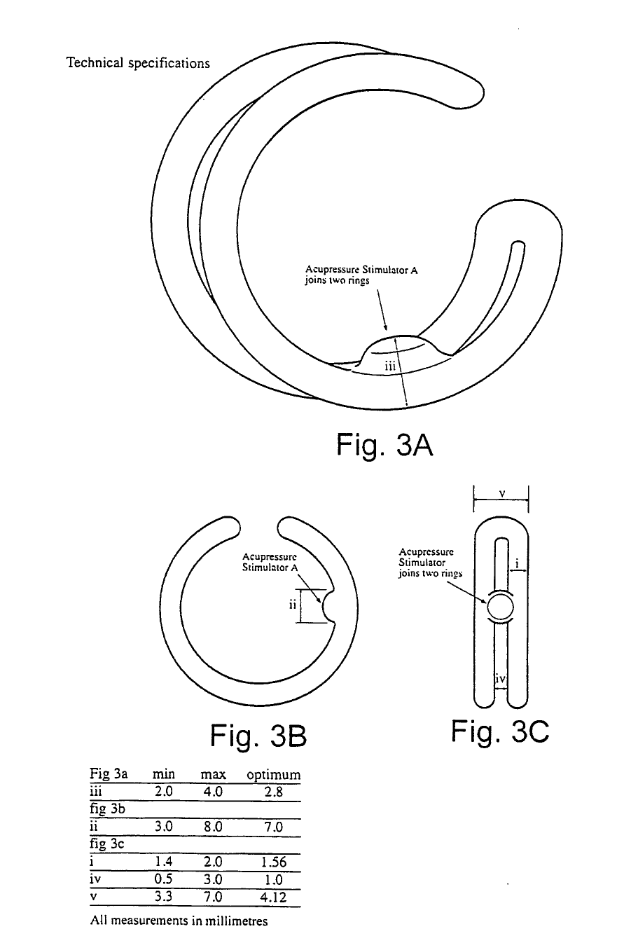

In a further embodiment of the present invention, the ring is a double ring, as shown and described in more detail in and in relation to FIG. 3 hereafter. In this case, the projection or projections preferably form a bridge or bridges between the two rings. If necessary, one or more other bridges, which do not project into the interior space defined by the rings, may be provided to hold the rings together. Further, if desired, there may be more than two rings held together by the projections(s) and/or other bridges.

If desired, the or each inwardly facing projection, or some of them, if there is more than one, may be matched with a corresponding outwardly facing projection or with a mark on the outer face of the ring, to assist the wearer to see where the inwardly facing projection(s) is or are located.

It will be appreciated that the distance that the projection or projections extend from the inner surface of the ring should be sufficient to exert the necessary pressure to achieve the desired acupressure effect, but not sufficient to prevent the wearer putting the ring on or to make the ring uncomfortable to wear. Ideally, each ring would be custom made for the individual, in which case this distance will be determined individually. However, this would be uneconomic in many cases, and so preformed rings are likely to be sold. In this case, we prefer that the distance that the or each projection extends from the inner surface of the ring should be from 1 to 4 mm, more preferably from 1 to 3.5 mm, still more preferably from 1.5 to 2.0 mm and most preferably from 1.6 to 2.0 mm.

The visual indicator is provided so that the wearer can locate that indicator on the back of the finger, which is easy to find with accuracy, and thus know that the projection will be in the correct position to contact the acupressure point. In a preferred aspect of the present invention, this visual indicator is provided by a break in the circumference of the ring. However, it may be provided by other means, for example an outwardly facing projection.

It is preferred that the ring should be adjustable for size to accommodate fingers of different sizes, and a preferred method of achieving this is to provide a break in the ring. Thus, if the break is suitably located, it may serve the dual functions of allowing for size adjustment and providing a visual indicator.

In another aspect, the invention provides an acupressure ring for use on the outer finger, and comprising a ring having a screw, the screw comprising a shank extending through the ring, said shank being attached at one end to a head portion and bearing a screw portion engaging with a corresponding screw extending through the ring, the head portion being movable by the screw shaft between a first position in which it is outside the outer surface of the ring and a second position in which it is closer to, adjacent or below the outer surface of the ring, means being provided to prevent the head portion moving further into the ring than said second position.

Preferably, the end of the shank remote from the head portion extends from 1.0 to 2.5 mm, more preferably from 1.5 to 2.0 mm, and most preferably about 1.6 mm, from the inner surface of the ring. The shank preferably has an enlarged portion at the end remote from the head, the enlarged portion preferably being removable.

The ring of the present invention may be made of any material from which conventional rings may be made. Clearly it should be of a material which is inert to the human skin, but beyond that, there is little restriction on the material used. Preferably the ring is made of a metal, such as silver, gold, platinum or titanium. However, it may also be made of another metal, such as copper, brass, zinc or steel, or it may be made of an organic material, such as wood, an organic polymer (e.g. Kevlar), plastic or rubber. It may also be made of ceramics or stones, including semi-precious stone, such as diamonds, emeralds, rubies, garnets, zircon, zirconium, opals, pearls, amethyst, agate, coral, jade, onyx, or sapphire. It may also be made of carbon fibre.

The material of which the ring is made may be solid or extruded to form a hollow ring band.

The invention is further illustrated with reference to the accompanying drawings, in which:

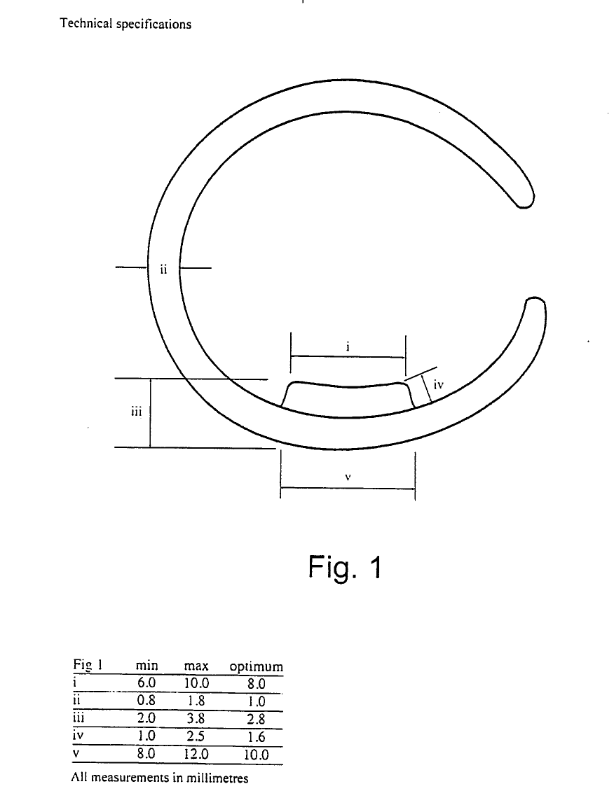

FIG. 1 shows a ring having a single projection in accordance with the present invention;

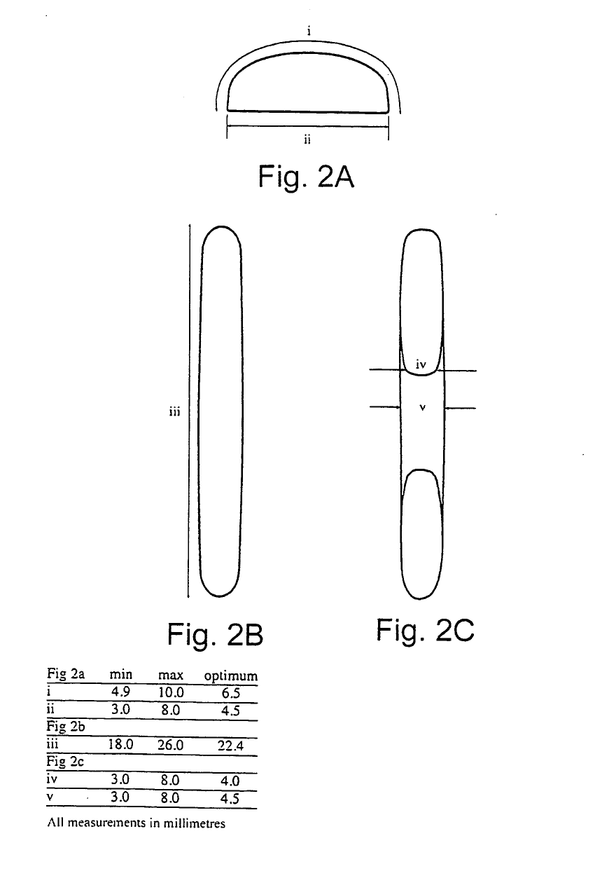

FIG. 2a shows a cross-section through a ring of the present invention with suggested dimensions;

FIGS. 2b and 2c show plan views of the ring of FIG. 2a;

FIGS. 3a, 3b and 3c show various views of a double ring in accordance with the present invention;

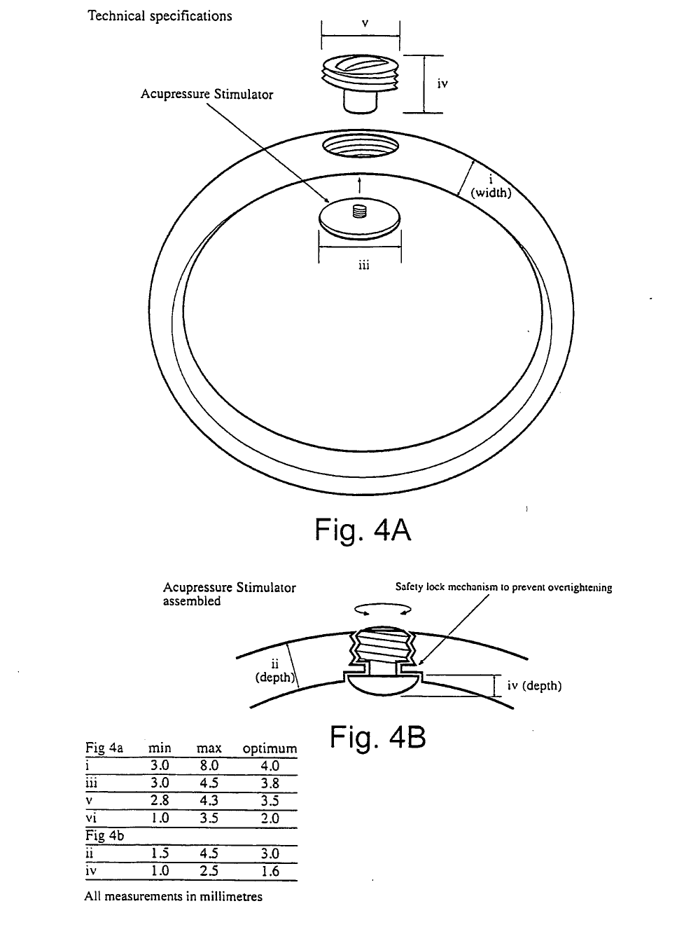

FIGS. 4a and 4b show an alternative embodiment of the ring of the present invention;

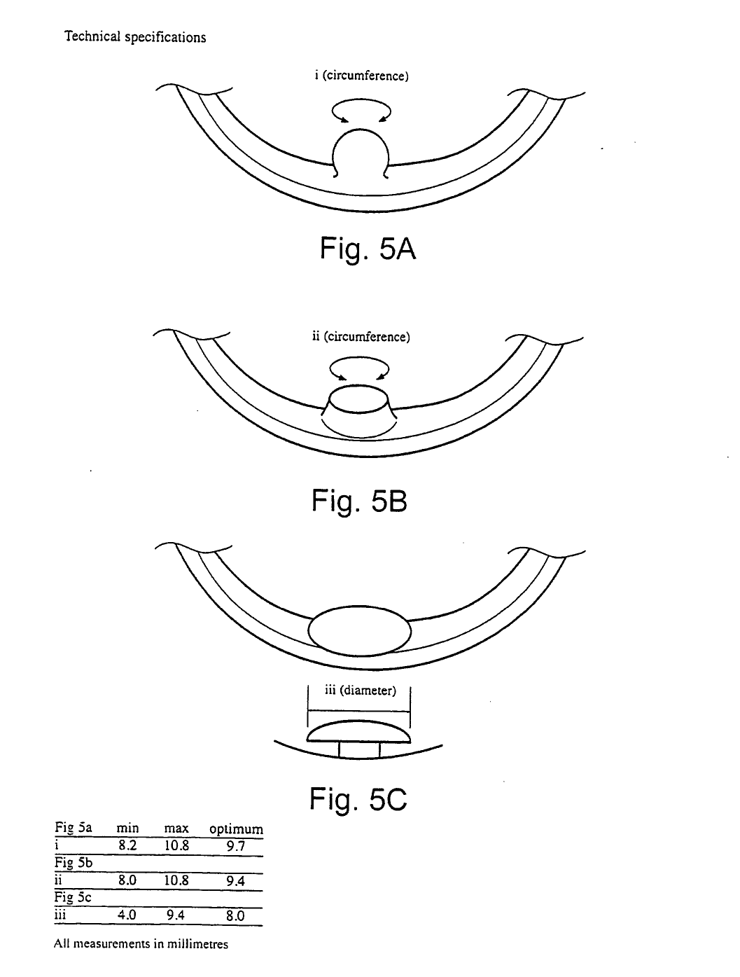

FIGS. 5a, 5b and 5c show partial views of rings in accordance with the present invention, having projections of different shapes; and

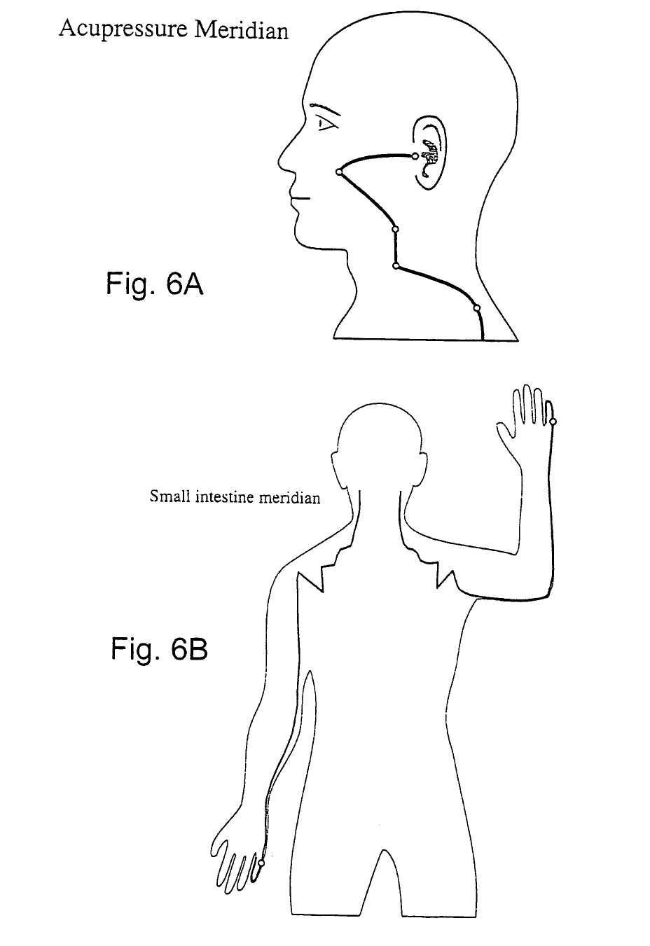

FIGS. 6 & 7 show the approximate location of the meridian lines relevant to the present invention.

Referring to FIGS. 1, 3 and 4, there is shown an acupressure ring in accordance with the present invention having a ring body and an inwardly extending projection. The projection is substantially rectangular in cross-section where it contacts the finger of the wearer, and extends inwardly about 1.6 mm from the inner surface of the ring body. The ring has a break in a location about 90°±10° from the mid-point of the projection.

FIGS. 2a, 2b and 2c show a ring of the present invention, giving suggested and preferred dimensions. The projection is hidden in the views shown.

FIG. 3 shows a double ring, in which the two ring portions are joined at the ends and are bridged by a single projection of substantially circular cross-section.

FIG. 4 shows an embodiment of the invention in which a ring has a threaded aperture extending between the outer and inner surfaces. Fitting into the aperture is an acupressure stimulator having a head, a shank and a screw threaded portion. The stimulator is prevented from moving too far by shoulders within the aperture (FIG. 4b).

Alternatively, FIGS. 5a, 5b and 5c show different forms of inwardly extending projection. That shown in FIG. 5a is of a generally spherical shape. Although not shown, this ring, as well as those of FIGS. 5b and 5c, still has a break in the ring, corresponding to the break in the ring of FIG. 1 and in a similar relative location.

FIG. 5b shows a partial view of a ring in which a projection has a truncated conical shape.

FIG. 5c shows a partial view of a ring in which a projection has a mushroom shape.

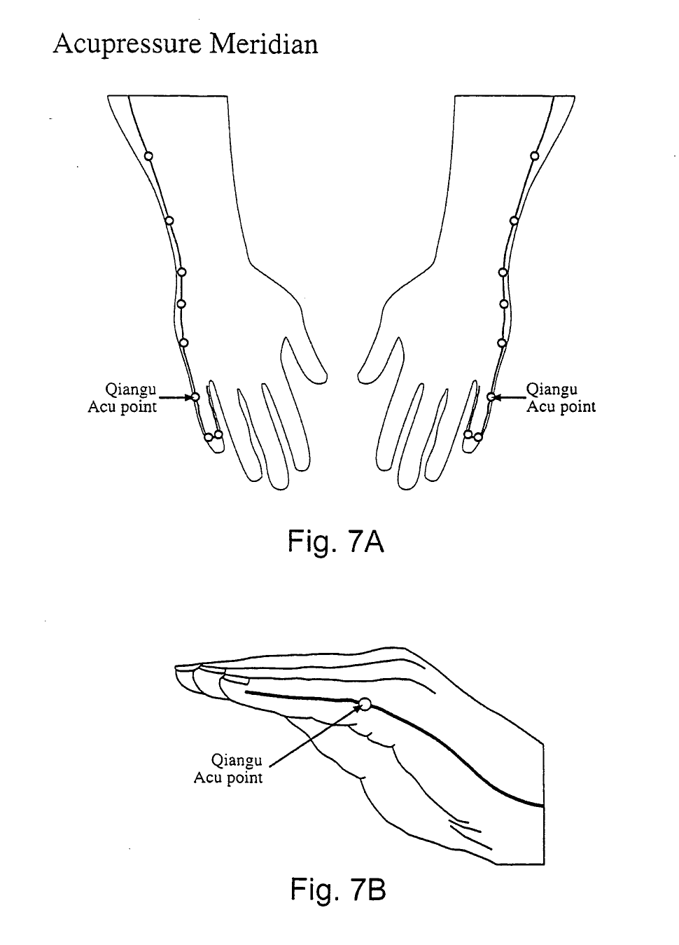

FIGS. 6 and 7 show the approximate location of the small intestine meridian lines and the qiangu acupressure points on the outer fingers.

1. An acupressure ring for use on the outer finger, and comprising a ring having a visual indicator visible when the ring is worn and a projection extending inwardly from the inner surface of the ring, the projection being approximately orthogonal to the visual indicator.

2. An acupressure ring according to claim 1, in which the mid-point of the projection is approximately 90° around the ring from the mid-point of the visual indicator, e.g. within ±200.

3. An acupressure ring according to claim 1 or claim 2, in which the cross section of the projection is round, cylindrical, conical, square or rectangular.

4. An acupressure ring according to any one of claims 1 to 3, in which there is a single projection.

5. An acupressure ring according to any one of claims 1 to 4, in which the ring is a double ring.

6. An acupressure ring according to claim 5, in which there is a bridging member between each of the rings.

7. An acupressure ring according to any one of the preceding claims, in which the visual indicator is provided by a break in the ring.

9. An acupressure ring according to any one of the preceding claims, in which the distance that the or each projection extends from the inner surface of the ring is from 1 to 4 mm.

10. An acupressure ring according to claim 9, in which said distance is from 1.6 to 2.0 mm.

11. An acupressure ring for use on the outer finger, and comprising a ring having a screw, the screw comprising a shank extending through the ring, said shank being attached at one end to a head portion and bearing a screw portion engaging with a corresponding screw extending through the ring, the head portion being movable by the screw shaft between a first position in which it is outside the outer surface of the ring and a second position in which it is closer to, adjacent or below the outer surface of the ring, means being provided to prevent the head portion moving further into the ring than said second position.

12. An acupressure ring according to claim 11, in which the end of the shank remote from the head portion extends from 1.0 to 2.5 mm, more preferably from 1.5 to 2.0 mm, from the inner surface of the ring.

13. An acupressure ring according to claim 11 or claim 12, in which the shank has an enlarged portion at the end remote from the head.

14. An acupressure ring according to claim 13, in which the enlarged portion is removable.
